(19) United States
(12) Patent Application Publication
(10) Pub. No.: US 2026/0083967 A1
(43) Pub. Date: Mar. 26, 2026

(54) TRANSVENUS REVERSIBLE ELECTROPORATION

(71) Applicant: Northwestern University, Evanston, IL (US)

(72) Inventors: Rishi K. Arora, Chicago, IL (US); David A. Johnson, Romeoville, IL (US)

(21) Appl. No.: 19/111,123

(22) PCT Filed: Sep. 12, 2023
(86) PCT No.: PCT/US2023/073941

§ 371 Date: Mar. 12, 2025

Related U.S. Application Data
(60) Provisional application No. 63/406,538, filed on Sep. 14, 2022.

(51) Int. Cl. A61N 1/32; A61N 1/05; A61N 1/37

(52) U.S. Cl. CPC A61N 1/327; A61N 1/056; A61N 1/3702

(57) ABSTRACT

A system to perform reversible transvenous electroporation includes an electroporation generator and a controller operably coupled to the electroporation generator. The controller is configured to instruct the electroporation generator to generate a voltage signal to perform electroporation, where the voltage signal has a predetermined range of voltages and a predetermined range of pulse widths to ensure that the electroporation is reversible, and where the voltage signal is biphasic or monophasic. The controller also delivers the voltage signal to a catheter to perform the electroporation, where the catheter includes one or more electrodes through which the voltage signal is delivered.

### CROSS-REFERENCE TO RELATED APPLICATION

The present application claims the priority benefit of U.S. Provisional Patent App. No. 63/406,538 filed on Sep. 14, 2022, the entire disclosure of which is incorporated herein by reference.

### REFERENCE TO GOVERNMENT RIGHTS

This invention was made with government support under Grant No. 5R35HL161249-02 awarded by the National Institutes of Health. The government has certain rights in the invention.

### BACKGROUND

Biological membranes are organized into lipid bilayers that separate the cytosol from extracellular fluid. These semi permeable barriers only allow diffusion of certain small uncharged or hydrophobic molecules and some large via channels or pumps. Although the cell membrane is structurally stable, the fatty acids are held together by weak van der Waals forces giving the membrane its semipermeable properties. If an electrical field of sufficient strength is applied, water dipoles on either side of the bilayer reorient to the field, forming hydrophobic pores or nanopores. This process is referred to as electroporation. Traditional electroporation is irreversible in that the permeabilization leads to cell death.

### SUMMARY

An illustrative system to perform reversible transvenous electroporation includes an electroporation generator and a controller operably coupled to the electroporation generator. The controller is configured to instruct the electroporation generator to generate a voltage signal to perform electroporation, where the voltage signal has a predetermined range of voltages and a predetermined range of pulse widths to ensure that the electroporation is reversible, and where the voltage signal is biphasic or monophasic. The controller also delivers the voltage signal to a catheter to perform the electroporation, where the catheter includes one or more electrodes through which the voltage signal is delivered.

In an illustrative embodiment, the system also includes a pacing system configured to generate a pacing signal, where the pacing rate is faster than a duration between pulses of the voltage signal. In one embodiment, the pacing system includes a pacing catheter to detect a heart pace of a patient upon which the electroporation is being performed. In another embodiment, the system includes a pacing isolator in communication with the pacing catheter. In another illustrative embodiment, the pacing system uses a blanking period in combination with the pacing signal to control the duration between electroporation pulses.

In another embodiment, the catheter includes a plurality of spines, and each spine in the plurality of spines includes a plurality of electrodes. In an illustrative embodiment, the plurality of electrodes on each spine alternate between positive and negative electrodes. In another embodiment, a first electrode of the plurality of electrodes receives the voltage signal and a second electrode of the plurality of electrodes is a return path for the voltage signal. In such an embodiment, the first electrode is adjacent to the second electrode. In another embodiment, the controller limits the voltage signal to 300 Volts/centimeter or less to ensure that the electroporation is reversible. In another embodiment, the controller limits the pulse duration of the voltage signal to 20 milliseconds or less to ensure that the electroporation is reversible.

An illustrative method for performing reversible electroporation includes generating, by an electroporation generator, a voltage signal to perform electroporation. The voltage signal has a predetermined range of voltages and a predetermined range of pulse widths to ensure that the electroporation is reversible, and the voltage signal is monophasic or biphasic. The method also includes delivering the voltage signal to a catheter to perform the electroporation, where the catheter includes one or more electrodes through which the voltage signal is delivered.

In one embodiment, the method includes generating, by a pacing system configured, a pacing signal such that the pacing rate is faster than a duration between pulses of the voltage signal. In another embodiment, the method includes detecting, by a pacing catheter of the pacing system, a heart pace of a patient upon which the electroporation is being performed. In another embodiment, the method includes using, by the pacing system, a blanking period in combination with the pacing signal to control a duration between electroporation pulses.

In an illustrative embodiment, the catheter includes a plurality of spines, and each spine in the plurality of spines includes a plurality of electrodes. In another embodiment, the plurality of electrodes on each spine alternate between positive and negative electrodes. In one embodiment, the method can include delivering the voltage signal to a first electrode of the plurality of electrodes such that a second electrode of the plurality of electrodes is a return path for the voltage signal. The method can also include limiting, by a controller, the voltage signal to 300 Volts/centimeter or less to ensure that the electroporation is reversible. The method can further include limiting, by a controller, the pulse duration of the voltage signal to 20 milliseconds or less to ensure that the electroporation is reversible.

Other principal features and advantages of the invention will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1A depicts a comparison of an injected rejection (PLA) and an uninjected region (LAA) in accordance with an illustrative embodiment.

FIG. 1B depicts NOX2 short hairpin (shRNA) experimental results in accordance with an illustrative embodiment.

FIG. 2A depicts the GFP expression results of electroporation on a subject in accordance with an illustrative embodiment.

FIG. 2B is another view depicting the localized region of electroporation in accordance with an illustrative embodiment.

FIG. 3 depicts a system for performing transvenous electroporation in accordance with an illustrative embodiment.

FIG. 4 is a comparison of a biphasic waveform and a monophasic waveform in accordance with an illustrative embodiment.

FIG. 5 is a timeline of electroporation performed using a biphasic waveform in accordance with an illustrative embodiment.

FIG. 6 depicts experimental results showing parameters used to perform successful transvenous reversible electroporation in accordance with an illustrative embodiment.

FIG. 7 depicts right and left atria parameters for the test subjects in accordance with an illustrative embodiment.

FIG. 8 depicts a computing system in direct or indirect communication with a network in accordance with an illustrative embodiment.

### DETAILED DESCRIPTION

Traditional electroporation procedures are irreversible in nature. Irreversible electroporation creates nanopores to facilitate focal destruction of aberrant cells. Voltages are delivered to the cells based on a lethal threshold, which is a metric of the susceptibility of a certain tissue or cell type to irreversible electroporation (IRE) induced cell death. While based on many factors, for most tissues, this lethal threshold is between 300 and 1000 V/cm when 100 pulses are applied. The end result of IRE is a targeted pulsed electric field (PEF) creation of lethal nanopores resulting in cell membrane compromise and cell death.

Described herein are methods and systems for performing reversible electroporation. Reversible electroporation (RE) creates nanopores that facilitate the delivery of a gene or drug into a cell for treatment, diagnosis, prevention, etc. An electrical field is applied and transient nanopores are formed, allowing Transgene-containing plasmids to enter the cytoplasm of the cell. Unlike IRE, the electricity applied is below the cells'lethal threshold, therefore damage to the phospholipid bilayer can be repaired and cellular death is avoided. The result of RE is the targeted creation of nanopores to enable drug/gene delivery into the cytoplasm of a cell and leave the cell membrane intact, thereby avoiding cellular death.

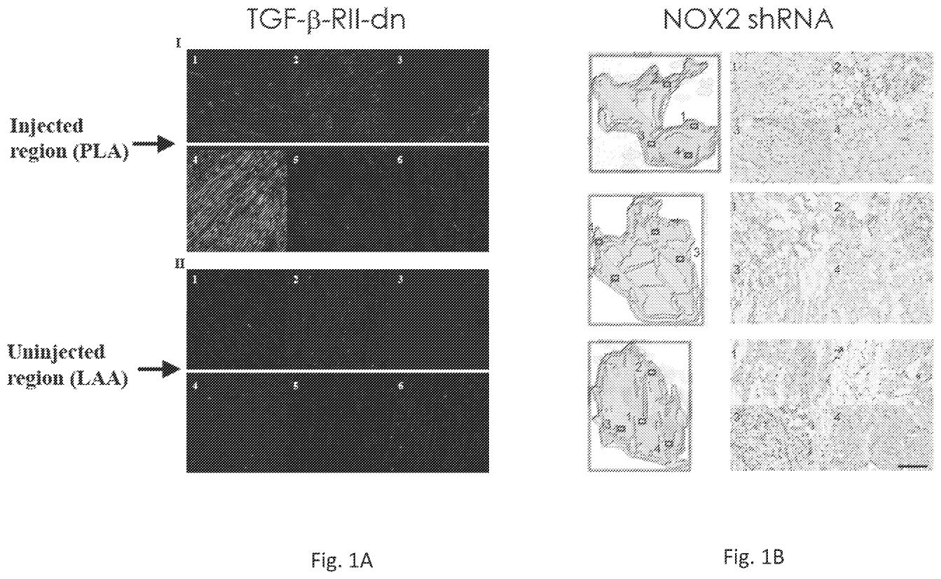

The inventors have explored both epicardial and transvenous electroporation gene treatments. Epicardial experiments were performed, and involved therapeutic genes injected sub-epicardially with 4-6 injections per atrial region. Electroporation was performed to facilitate gene transfer, with electroporation parameters of 150-200 V/cm, 10 ms pulse width, 8-10 pulses, and 1 second inter-pulse duration. Using these techniques, gene transfers were performed over 100 times using electroporation. Evidence of gene expression and efficacy was noted up to 6 months after the electroporation was performed. There was also evidence of reversal of atrial fibrillation (AF) disease. There was no evidence of overt toxicity at any point up to 6-8 months out from the procedures. FIG. 1 depicts generally homogenous gene expression that resulted from electroporation experiments. Specifically, FIG. 1A depicts a comparison of an injected rejection (PLA) and an uninjected region (LAA) in accordance with an illustrative embodiment. FIG. 1B depicts NOX2 short hairpin (shRNA) experimental results in accordance with an illustrative embodiment.

The inventors have also developed a new transvenous approach for electroporation (endocardial) gene delivery. In on embodiment, the system uses catheters (e.g., FirMap (Abbott), Orion (BSC), Constellation (BSC), etc.) for the electroporation, along with a generator and a pacer. To test this approach, a Coomassie blue dye injection procedure was performed. Specifically, in two subjects, the coronary sinus was cannulated via a jugular venous approach. In one subject, a catheter (64 electrodes) was advanced into the high right atrium via femoral venous approach. In the second subject, a catheter was advanced into the left atrium via transseptal puncture. In both subjects, balloon occlusion was performed in the proximal coronary sinus, followed by coronary sinus injection of Coomassie blue dye. While injection was being performed, electroporation was performed simultaneously in the right or left atrium via the catheters. The electroporation was performed at 200 V/cm with a pulse duration of 1 millisecond (ms), with 20 pulses used, and a 1 second interval between pulses. The atria of both subjects was later examined, and the Coomassie blue was found only in the atrium where electroporation was performed, with no dye present in the other atrium. This confirms that the electroporation process worked as intended.

In a third subject, 1.5 milligrams (mg) of Green Fluorescent Protein-(GFP-) expressing plasmid (under control of a CMV promoter) was diluted up to 20 milliliters (ml) and injected in the coronary sinus. Simultaneous electroporation was performed as described in the high right atrium (right atrial free wall) with a FirMap catheter, as described for Coomassie blue. The gene injection and electroporation sequence was repeated three times. After 3 days, the heart of the third subject was analyzed. The electroporated high right atrium (high and mid right atrial free wall) and non-electroporated right atrium (low right atrial free wall, right atrial appendage, posterior right atrium) and non-electroporated left atrium were examined for GFP expression using fluorescence microscopy and western blotting. Based on the analysis, GFP expression was noted only in the electroporated parts of the right atrium (high and mid right atrial free wall) with no evidence of GFP in the non-electroporated right or left atrium.

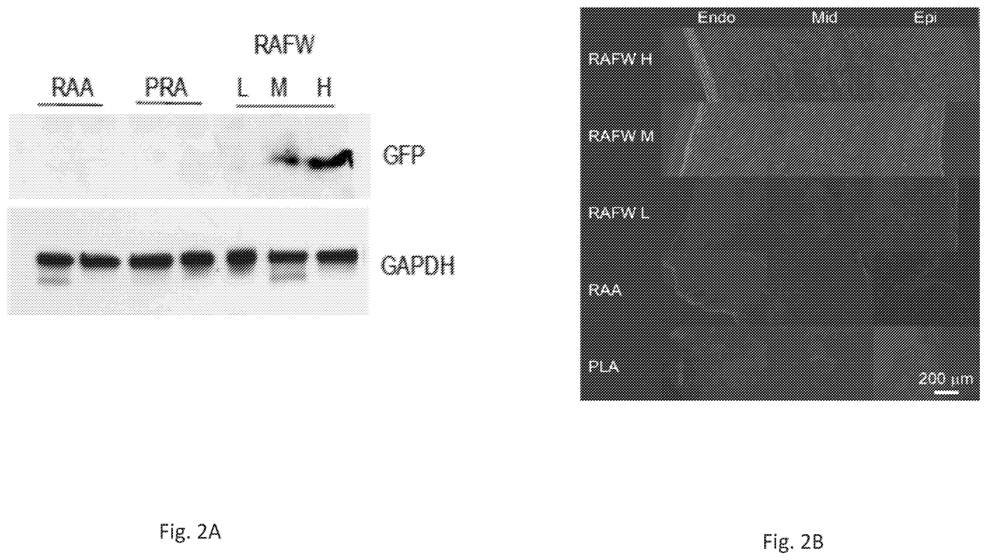

FIG. 2A depicts the GFP expression results of electroporation on a subject in accordance with an illustrative embodiment. As shown, GFP expression is localized to the region of electroporation. In FIG. 2A, RAFW refers to right atrial free well, H is high, M is mid, L is low, RAA is the right atrial appendage, and PRA is the posterior right atrium. FIG. 2B is another view depicting the localized region of electroporation in accordance with an illustrative embodiment. In FIG. 2B, PLA is the posterior left atrium, endo refers to endocardium, Mid is the mid myocardium, and Epi is the epicardium. As shown in FIGS. 2A and 2B, GFP expression was transmural (i.e. epi to endocardial expression).

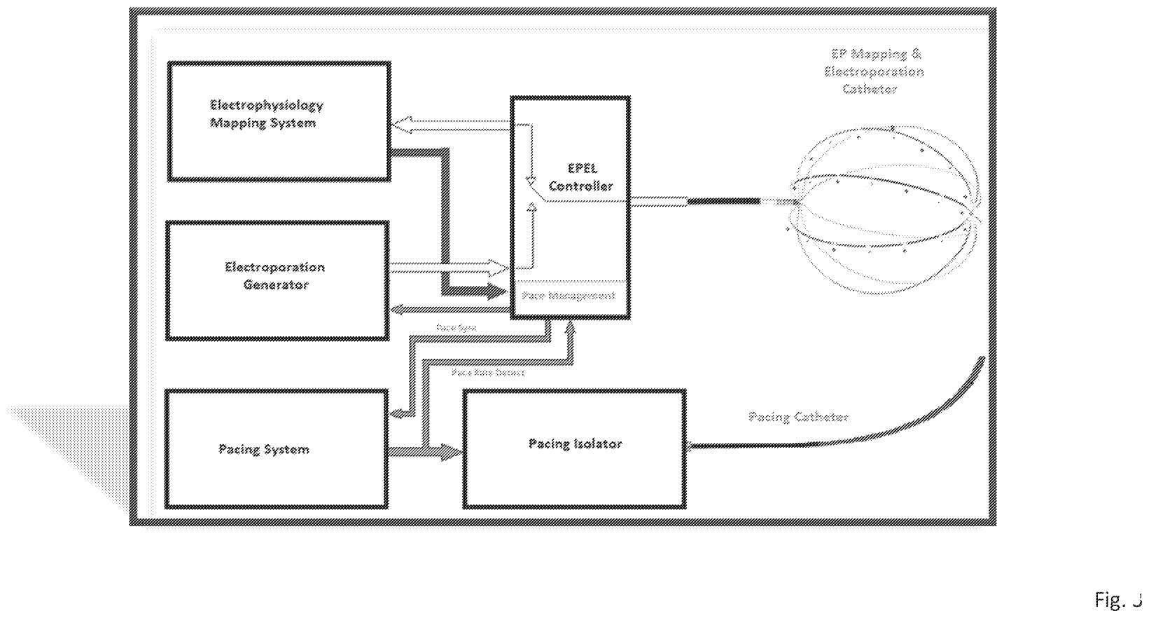

FIG. 3 depicts a system for performing transvenous electroporation in accordance with an illustrative embodiment. The system includes an electroporation generator to deliver a pulsed electric field. Any type of electroporation generator can be used. The system also includes a controller with pace management. In one embodiment, the controller is an Extra Packages for Enterprise Linux (EPEL) controller. Alternatively, any other type of controller may be used. The system further includes a pacing system, a pacing isolator, a pacing catheter, an electrophysiology (EP) mapping and electroporation catheter, and an electrophysiology mapping system. In alternative embodiments, the system can include fewer, additional, and/or different components.

As shown, the catheter includes a plurality of electrodes, and the polarities of the electrodes alternates between neighboring electrodes. Pulses are applied across pairs of electrodes in contact with the heart. As an example there may be 2, 3, or more electrodes with the polarity of each neighboring electrodes voltage potential to be the opposite of its neighbors. As an example, electrode 1 is applied a positive voltage potential, where electrode 2 is a neighboring electrode and it is the voltage/current return path for the voltage applied in electrode 1. Electrode 3 is a neighbor to electrode 2 and therefore has a positive voltage potential applied to it, electrode 4 has electrode 3 as its neighbor and therefore it is the voltage/current potential return path for the voltage potential applied in electrode 3, and so on. In one embodiment, the catheter uses 64 electrodes. Alternatively, a different number may be used such as 16, 32, 128, etc.

The inventors have, through experimentation, developed ranges for a number of different electroporation parameters such that the safe and effective reversible transvenous electroporation gene delivery can be performed. In an illustrative embodiment, it was determined that the pulse voltage amplitude can range from 10 V/cm to 300 V/cm in one embodiment. A range for pulse duration was found to be 0.1 ms to 20 ms. Pulse shape was also considered. A pulse may be a slope of a defined rate, which is a series of sequential step pulses each defined for a specific duration such as 200V/cm for 0.1 ms followed by a +50V pulse at 5 ms followed by a 0.1 ms pulses at −200V/cm and at −50V/cm. Repetition of the start of each electroporation pulse may be at a rate which is in sync with the natural rhythm of the heart or triggered by another external device such as a pacing generator. A software algorithm is used to ensure the delivery to occur with the heart beat and in sync with the input trigger but no less than 250 ms, else it will delay the next pulse to be delivered to the subsequent timing to be in sync with the heart beat and/or external trigger.

The pacing system is used to control the timing of delivery of the electroporation pulses to avoid ventricular fibrillation. In one embodiment, the pulse from the pacing system is faster than the patient's actual pulse. It is desirable to perform the electroporation at a constant rate. As one example, the rate can be once per second. In such an embodiment, the pacing system pulse may be 400 ms, which means that the electroporation cannot occur at every pacing system pulse (i.e., it would then occur more than the desired once per second). The pulse from the pacing system can be repeated multiple times and/or used in conjunction with a pause (or blanking period) by the pacing system. For example, the system may perform a first electroporation and then wait for 2 pacing system pulses (˜800 ms in the example above) along with a blanking period of ˜200 ms prior to performing the second electroporation at ˜1 second after the first electroporation. In an alternative embodiment, using the same example, the pacing system may perform a first electroporation, wait for a next pulse of the pacing system to occur (at ˜400 ms), and then initiate a blanking period of 600 ms such that the second electroporation occurs at ˜1 second after the first. In alternative embodiments, different pulse timing may be used by the pacing system (e.g., 200 ms, 300 ms, 500 ms, 700 ms, etc. in between pulses), and/or different blanking period lengths may be used (e.g., 800 ms, 700 ms, 500 ms, 400 ms, 300 ms, etc.).

To perform electroporation at a constant rate, one can use any combination of a pacing rate and/or a blanking period. As such, either or both of the pacing rate and blanking period parameters can be adjusted to control the electroporation at a constant rate. When using pacing rate, electroporation duration/rate can be programmed to be a ‘multiple’ or a ‘ratio’ of the pacing rate e.g. if pacing rate is 400 msec, then one should be able to obtain an electroporation rate of 1200 msec by specifying a 1:3 between electroporation rate/pacing rate.

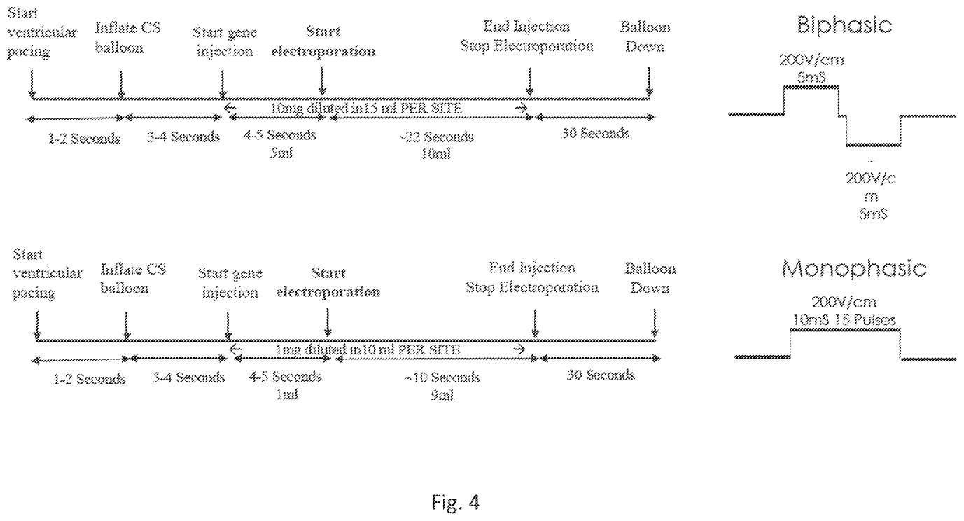

In an illustrative embodiment, the system can utilize a bi-phasic waveform to perform the electroporation. The bi-phasic waveform includes the applied voltage divided into two phases of opposite polarity. The first phase can be identical to a monophasic waveform, although typically of shorter duration, and the second phase is discharged at the opposite polarity for a set time, which can be equal to the length of the first phase or different therefrom, depending on the implementation. FIG. 4 is a comparison of a biphasic waveform and a monophasic waveform in accordance with an illustrative embodiment. Specifically, FIG. 4 depicts timelines of the waveforms that illustrate a start of ventricular pacing (e.g., by the pacing system), balloon inflation, the start of gene injection, the start of electroporation, an end of gene injection and a stop to electroporation, and balloon deflation.

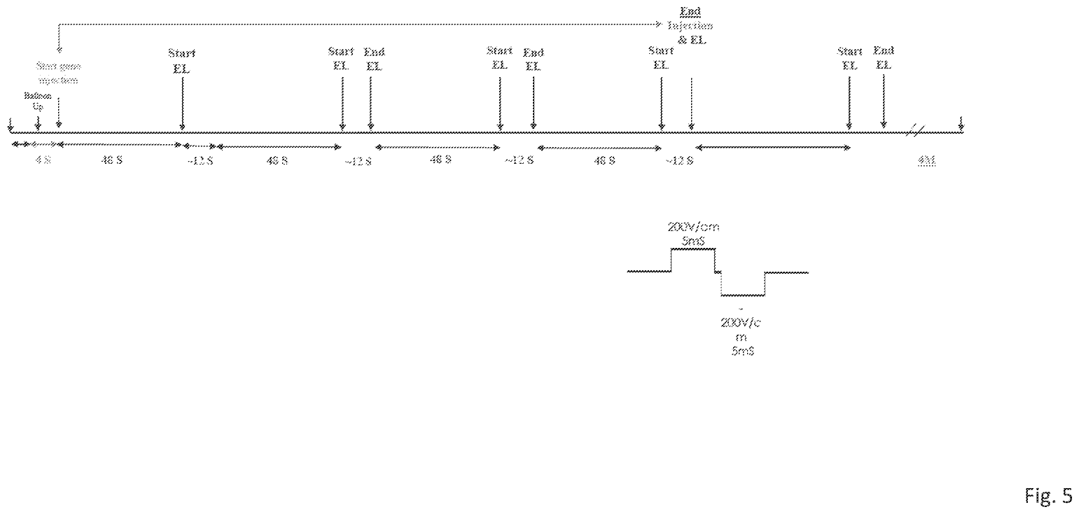

FIG. 5 is a timeline of electroporation performed using a biphasic waveform in accordance with an illustrative embodiment. The electroporation of FIG. 5 was performed with four minutes of injection at 10 mg in 40 mL per region. Each electroporation train was 10 pulses and is bipolar at 200 V/cm. In alternative embodiments, a different amount of substance may be injected, the number of pulses can be different, and/or the voltage can be changed. The bipolar electroporation was repeated 3 times at total positions.

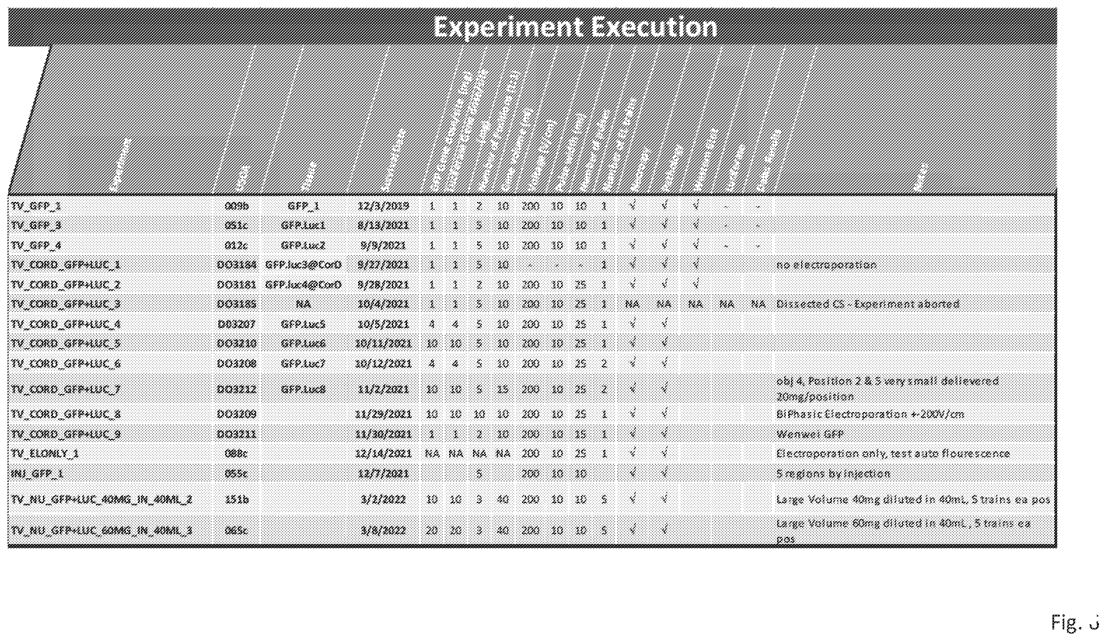

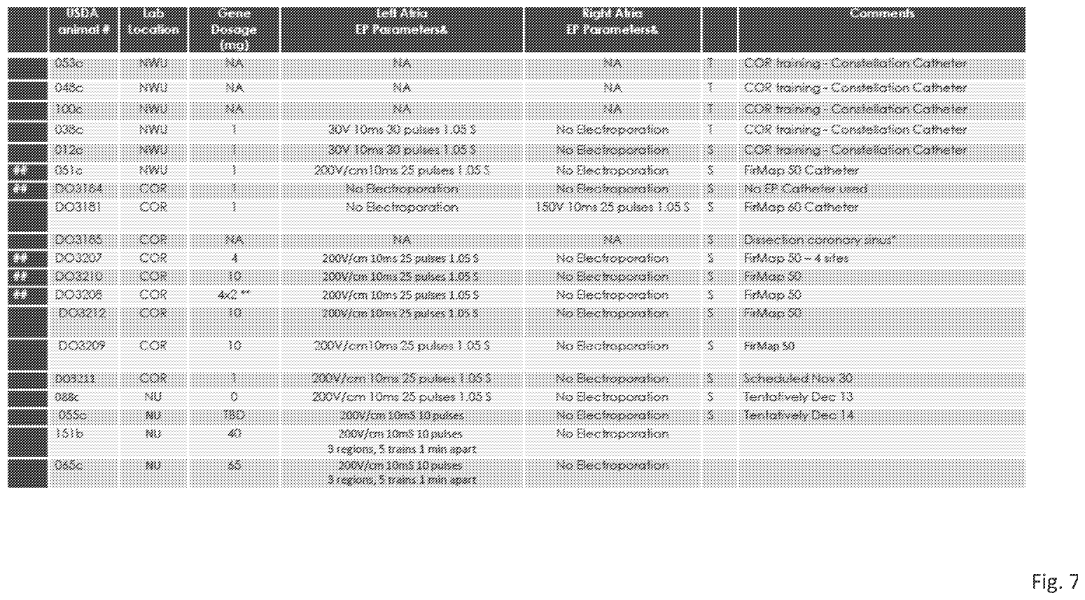

The proposed techniques are the first of their kind for use on the atria. These techniques can also be used on the ventricle. Unlike traditional procedures, the electroporation described herein is a reversible low energy approach that does not permanently damage the cells being targeted. The inventors have developed the ideal system and operating parameters to perform the reversible transvenous electroporation procedures described herein. Specifically, the inventors have developed gene parameters including gene dosage, gene volume, and rate of gene injection. The inventors have also developed electroporation parameters such as voltage/cm, pulse width, number of pulses, inter-pulse duration, and polarity (e. g, biphasic). The inventors have also developed timing parameters such as gene dwell time after the end of electroporation, scenarios in which electroporation precedes gene injection, and scenarios in which gene injection precedes electroporation. FIG. 6 depicts experimental results showing parameters used to perform successful transvenous reversible electroporation in accordance with an illustrative embodiment. FIG. 7 depicts right and left atria parameters for the test subjects in accordance with an illustrative embodiment.

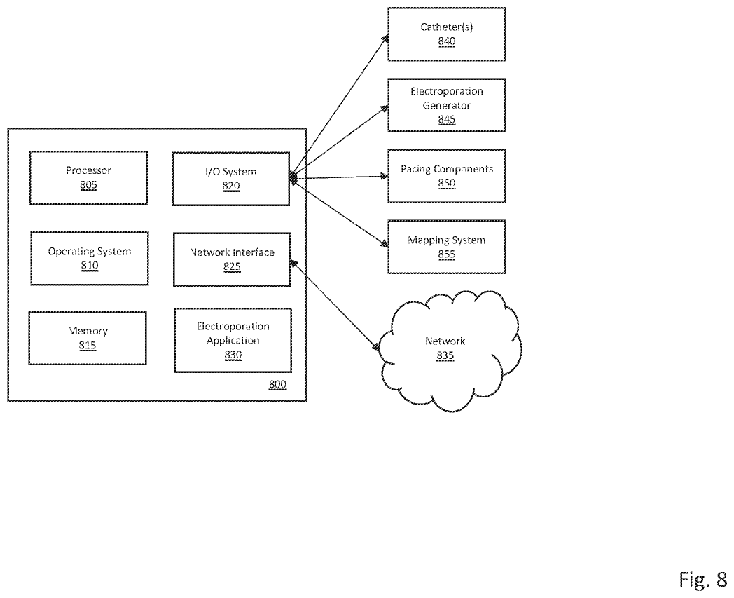

In an illustrative embodiment, any of the operations described herein can be performed by a computing system that includes a processor, memory, user interface, transceiver, etc. The memory can be used to store computer-readable instructions that, upon execution by the processor, implement the operations described herein. The computing system can be incorporated into any of the system components described herein, and/or a separate computing system may be used. As an example, FIG. 8 depicts a computing system 800 in direct or indirect communication with a network 835 in accordance with an illustrative embodiment. The computing system 800 includes a processor 805, an operating system 810, a memory 815, an input/output (I/O) system 820, a network interface 825, and an electroporation application 830. In alternative embodiments, the computing system 800 may include fewer, additional, and/or different components. The components of the computing device 800 communicate with one another via one or more buses or any other interconnect system. In an illustrative embodiment, the computing system 800 can act as the system controller depicted in FIG. 3, and can interact with and control various other system components to perform the electroporation described herein.

The processor 805 of the computing system 800 can be in electrical communication with and used to perform any of the operations described herein, such as controlling a pacing system to be in sync with a heart, delivering an electrical pulse, controlling a strength of the pulse, controlling a number of pulses, controlling a duration of pulses, controlling an amount of substance introduced into a cell, controlling balloon inflation and deflation, etc. The processor 805 can be any type of computer processor known in the art, and can include a plurality of processors and/or a plurality of processing cores. The processor 805 can include a controller, a microcontroller, an audio processor, a graphics processing unit, a hardware accelerator, a digital signal processor, etc. Additionally, the processor 805 may be implemented as a complex instruction set computer processor, a reduced instruction set computer processor, an x86 instruction set computer processor, etc. The processor 805 is used to run the operating system 810, which can be any type of operating system,

The operating system 810 is stored in the memory 815, which is also used to store programs, algorithms, network and communications data, peripheral component data, the electroporation application 830, and other operating instructions. In an illustrative embodiment, the memory 815 stores computer-readable instructions that can perform any of the operations described herein. The computer-readable instructions can be executed by the processor 805 to perform the operations. The memory 815 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 820, or user interface, is the framework which enables users (and peripheral devices) to interact with the computing system 800. The I/O system 820 can include one or more keys or a keyboard, one or more buttons, one or more displays, a speaker, a microphone, etc. that allow the user to interact with and control the computing system 800. The I/O system 820 also includes circuitry and a bus structure to interface with peripheral computing components such as power sources, sensors, etc.

As shown, the I/O system 820 is in direct or indirect communication with various system components such as one or more catheters 840, an electroporation generator 845, pacing components 850, and a mapping system 855. The one or more catheters can include the EP mapping and electroporation catheter described herein, and one or more pacing catheters used to maintain the correct heart pacing. The electroporation generator 845 can be any type of electrical signal generator known in the art, such as a modified ECM 830 electroporation generator, a modified BTX Gemini X2 electroporation generator, etc. The pacing components 850 can include a pacing system and a pacing isolator. Any type of pacing system and/or pacing isolator known in the art may be used. The mapping system 855 can be any type of EP mapping system or algorithm known in the art.

The network interface 825 includes transceiver circuitry (e.g., one or more transmitters and one or more receivers) that allows the computing system 800 to transmit and receive data to/from other devices such as user device(s), remote computing systems, servers, other system components, websites, etc. The network interface 825 enables communication through the network 835, which can be one or more communication networks. The network 835 can include a cable network, a fiber network, a cellular network, a wi-fi network, a landline telephone network, a microwave network, a satellite network, etc. The network interface 825 also includes circuitry to allow device-to-device communication such as near field communication (NFC), Bluetooth® communication, etc.

The electroporation application 830 can include hardware, software, and algorithms (e.g., in the form of computer-readable instructions) which, upon activation or execution by the processor 805, performs any of the various operations described herein such as controlling a pacing system to be in sync with a heart detecting a pace rate and synching with the detected rate, delivering an electrical pulse, controlling a strength of the pulse, controlling a number of pulses, controlling a duration of pulses, controlling an amount or type of substance introduced into a cell, controlling balloon inflation and deflation, etc. The electroporation application 830 can utilize the processor 805 and/or the memory 815 as discussed above. For example, all or a portion of electroporation application 830 can be stored in the memory 815, and the processor 805 can be used to execute any of the operations stored in the electroporation application 830.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A system to perform reversible transvenous electroporation, the system comprising:
an electroporation generator; and
a controller operably coupled to the electroporation generator, wherein the controller is configured to:
instruct the electroporation generator to generate a voltage signal to perform electroporation, wherein the voltage signal has a predetermined range of voltages and a predetermined range of pulse widths to ensure that the electroporation is reversible, and wherein the voltage signal is monophasic or biphasic; and
deliver the voltage signal to a catheter to perform the electroporation, wherein the catheter includes one or more electrodes through which the voltage signal is delivered.

2. The system of claim 1, further comprising a pacing system configured to generate a pacing signal, wherein the pacing rate is faster than a duration between pulses of the voltage signal.

3. The system of claim 2, wherein the pacing system includes a pacing catheter to detect a heart pace of a patient upon which the electroporation is being performed.

4. The system of claim 3, further comprising a pacing isolator in communication with the pacing catheter.

5. The system of claim 2, wherein the pacing system uses a blanking period in combination with the pacing signal to control the duration between electroporation pulses.

6. The system of claim 1, wherein the catheter includes a plurality of spines, and wherein each spine in the plurality of spines includes a plurality of electrodes.

7. The system of claim 6, wherein the plurality of electrodes on each spine alternate between positive and negative electrodes.

8. The system of claim 7, wherein a first electrode of the plurality of electrodes receives the voltage signal and wherein a second electrode of the plurality of electrodes is a return path for the voltage signal.

9. The system of claim 8, wherein the first electrode is adjacent to the second electrode.

10. The system of claim 1, wherein the controller limits the voltage signal to 300 Volts/centimeter or less to ensure that the electroporation is reversible.

11. The system of claim 1, wherein the controller limits the pulse duration of the voltage signal to 20 milliseconds or less to ensure that the electroporation is reversible.

12. A method for performing reversible electroporation, the method comprising:
generating, by an electroporation generator, a voltage signal to perform electroporation, wherein the voltage signal has a predetermined range of voltages and a predetermined range of pulse widths to ensure that the electroporation is reversible, and wherein the voltage signal is monophasic or biphasic; and
delivering the voltage signal to a catheter to perform the electroporation, wherein the catheter includes one or more electrodes through which the voltage signal is delivered.

13. The method of claim 12, further comprising generating, by a pacing system configured, a pacing signal, wherein the pacing rate is faster than a duration between pulses of the voltage signal.

14. The method of claim 13, further comprising detecting, by a pacing catheter of the pacing system, a heart pace of a patient upon which the electroporation is being performed.

15. The method of claim 13, further comprising using, by the pacing system, a blanking period in combination with the pacing signal to control a duration between electroporation pulses.

16. The method of claim 12, wherein the catheter includes a plurality of spines, and wherein each spine in the plurality of spines includes a plurality of electrodes.

17. The method of claim 16, wherein the plurality of electrodes on each spine alternate between positive and negative electrodes.

18. The method of claim 17, further comprising delivering the voltage signal to a first electrode of the plurality of electrodes such that a second electrode of the plurality of electrodes is a return path for the voltage signal.

19. The method of claim 12, further comprising limiting, by a controller, the voltage signal to 300 Volts/centimeter or less to ensure that the electroporation is reversible.

20. The method of claim 12, further comprising limiting, by a controller, the pulse duration of the voltage signal to 20 milliseconds or less to ensure that the electroporation is reversible.
